(19) United States
(12) Patent Application Publication
(10) Pub. No.: US 2020/0345923 A1
(43) Pub. Date: Nov. 5, 2020

(54) MANUAL CENTRIFUGAL DEVICE AND SYSTEM COMPRISING THE SAME, AND METHOD FOR SEPARATING BLOOD

(71) Applicant: National Taiwan University, Taipei (TW)

(72) Inventors: Chien-Fu Chen, Taipei (TW); Chao-Hsuan Liu, Taipei (TW)

(21) Appl. No.: 16/706,945

(22) Filed: Dec. 9, 2019

(30) Foreign Application Priority Data
May 3, 2019 (TW) 108115670

(51) Int. Cl. A61M 1/36; A61B 5/15

(52) U.S. Cl. CPC A61M 1/3693; A61B 5/150755

(57) ABSTRACT

Provided is a manual centrifugal device, including a rotating platform disposed with a rotating shaft, and a plurality of blood storage tubes fixed to the rotating platform at a regular intervals, wherein the rotating platform and the plurality of blood storage tubes rotate relative to the plane of placement during centrifugation. Further provided is a method for separating blood. The centrifugal device is driven manually to centrifugally separate blood cells and blood plasma, thereby greatly reducing the medical cost of blood examination and providing a more convenient and simple blood separation method for the low-resource areas.

### BACKGROUND

### 1. Technical Field

The present disclosure relates to centrifugal devices that do not require electric power, and more particularly, to a manual centrifugal device and a method of separating blood.

### 2. Description of Related Art

In order to make medical care more popular in developing countries where resources are scarce, the global medical community is actively investing in the development of “precise medical care,” including the point-of-care (POC) system. The system should be used for detection purposes in accordance with the World Health Organization (WHO)'s standard requirements of affordability, sensitivity, specificity, stability, ease of operation, fast, no need for large equipment and easiness to carry.

The composition of blood can be divided into blood cells and plasma, and plasma contains water, protein, metabolites, viruses, bacteria and other components. By analyzing the composition ratio of plasma in the blood, the health of the human body can be initially determined, so that plasma detection is widely used for disease diagnosis and treatment. Effective plasma separation can reduce the interference of non-tested substances, avoid sample contamination, and improve the accuracy of detection.

The conventional plasma separation technique uses a high-speed centrifugal device to obtain high-purity plasma by a relative centrifugal force generated in a high-speed environment. However, current centrifugal equipment requires a motor to generate the centrifugal force, such that not only the medical cost for the low-resource area is extremely burdensome, but also the power supply is a tough issue.

In view of the above, it is necessary to propose a centrifugal device that does not require electric drive, is simple and fast to operate, and maintains the accuracy of detection of plasma after separation to solve the problems faced by the low-resource area.

### SUMMARY

In order to solve the above problems, the present disclosure provides a manual centrifugal device for separating blood, comprising: a rotating platform; a rotating shaft disposed at the center of the rotating platform; and a plurality of blood storage tubes disposed at a regular interval from one another and restrictedly connected to the rotating platform at a distance from the rotating shaft, wherein the rotating platform and the plurality of blood storage tubes rotate around the rotating shaft during centrifugation.

In an embodiment of the present disclosure, the plurality of blood storage tubes are disposed on an upper surface of the rotating platform. For example, the rotating platform has a buckle groove, and the plurality of blood storage tubes are embedded in the buckle groove.

In an embodiment of the present disclosure, the plurality of blood storage tubes are straight tubes, e.g., rod-like. The plurality of blood storage tubes are disposed away from the rotating shaft at a distance of 1 cm to 20 cm, and along a radial direction of the rotating shaft. In another embodiment of the present disclosure, the plurality of blood storage tubes are disposed at an angle of less than 90 degrees, e.g., 30 degrees to 75 degrees, with respect to a radial direction of the rotating shaft.

In another embodiment of the present disclosure, the plurality of blood storage tubes are positioned at an outer edge of the rotating platform.

In an embodiment of the present disclosure, the manual centrifugal device further includes a plurality of sleeves disposed at an outer edge of the rotating platform to accommodate the plurality of blood storage tubes, wherein the plurality of sleeves are pivotally connected with the outer edge of the rotating platform at a position between an end thereof and the center of the sleeve.

In an embodiment of the present disclosure, the rotating platform includes a connecting portion for the rotating shaft and a plurality of rotating arms, which are equidistantly disposed along a radial direction of the rotating shaft and connected to the connecting portion.

In an embodiment of the present disclosure, each of the blood storage tubes has a volume of 1 μL to 1000 μL. In an embodiment of the present disclosure, the plurality of blood storage tubes are straight tubes having a diameter of 1 mm to 10 mm.

In an embodiment of the present disclosure, the rotating shaft includes a rolling bearing and a supporting member having a stud, and the stud is fixed in the rolling bearing.

In an embodiment of the present disclosure, the arm has a load of 50 grams to 500 grams.

In an embodiment of the present disclosure, the rotating shaft is a shaft center having a sliding bearing. The manual centrifugal device further includes a base including a crank handle disposed on the outer casing of the base, and a gear transmission mechanism mounted on the casing cavity of the base, wherein the gear transmission mechanism is connected to the crank handle, and the rotating shaft is disposed on the base and coupled with the gear transmission mechanism.

The present disclosure further provides a method of separating blood using the manual centrifugal device of the present disclosure, including the steps of: loading blood into at least one of the blood storage tubes; optionally closing openings of the blood storage tubes; connecting the blood storage tubes restrictedly to the rotating platform; and spinning the rotating platform of the manual centrifugal device manually to separate blood plasma and blood cells in the blood storage tubes.

In an embodiment of the present disclosure, the plurality of the blood storage tubes are fixed to an upper surface of the rotating platform. The rotating shaft includes a rolling bearing and a supporting member having a stud, and the stud is fixed at the center of the rolling bearing; and the manual centrifugal device has a rotation speed of 1000 rpm to 1200 rpm and a separation time of 3 minutes to 15 minutes.

In another embodiment of the present disclosure, the rotating shaft is a shaft center having a sliding bearing, and the centrifugal device has a rotation speed of 2000 rpm to 3000 rpm and a separation time of 1.5 minutes to 5 minutes.

The present disclosure further provides a system for analyzing a target protein in plasma of a patient in a low-resource area, the system including: the above-mentioned manual centrifugal device for obtaining plasma containing a target protein; and a trace detection device with a captured protein, wherein the captured protein specifically binds to the target protein in the plasma.

Since the centrifugal device of the present disclosure is not plugged-in, driven by a hand without using electric power, and centrifugally separates blood cells and plasma in the blood, the medical cost burden of the resource-poor area can be greatly reduced. The centrifugal device of the present disclosure rotates horizontally with respect to the plane of placement, or rotates the blood storage tube with respect to the plane at less than 90 degrees, e.g., less than 60 degrees, 45 degrees, 30 degrees or 15 degrees. Compared to vertical rotation, the centrifugation can eliminate the interference of gravity, and improve the yield and purity of the plasma, so that it meets the requirements of detection accuracy.

### BRIEF DESCRIPTION OF THE DRAWINGS

Embodiments of the present disclosure are illustrated by way of examples with reference to the accompanying drawings:

FIG. 1 is a schematic view showing a manual centrifugal device in a first embodiment of the present disclosure;

FIG. 2 is a schematic view showing an aspect of a blood storage tube of the manual centrifugal device in the first embodiment of the present disclosure;

FIG. 3 is a schematic view showing the manual centrifugal device in a second embodiment in a first embodiment of the present disclosure;

FIG. 4 is a schematic view showing an internal structure of the manual centrifugal device in the second embodiment of the present disclosure;

FIG. 5 is a schematic view showing the manual centrifugal device in a third embodiment of the present disclosure;

FIG. 6 is a schematic view showing an internal structure of the manual centrifugal device in the third embodiment of the present disclosure;

FIG. 7 is a schematic view showing an aspect of a blood storage tube of the manual centrifugal device in the third embodiment of the present disclosure;

FIGS. 8A and 8B are cross-sectional schematic views showing an aspect of a fixed blood storage tube of the manual centrifugal device of the present disclosure taken along line A-A′ in FIG. 1;

FIG. 9 is a schematic view showing an aspect of a blood storage tube of a fourth embodiment of the manual centrifugal device of the present disclosure;

FIG. 10 is a schematic view showing a fifth embodiment of the manual centrifugal device of the present disclosure;

FIGS. 11A to 11B are schematic cross-sectional views showing a stationary and centrifugal operation of a fifth embodiment of the manual centrifugal device of the present disclosure, respectively;

FIG. 12A is a graph showing changes in the position of the blood storage tube arrangement versus the yield of the manual centrifugal device in the second embodiment of the present disclosure;

FIG. 12B is a graph showing changes in the position of the blood storage tube arrangement versus the purity of the manual centrifugal device in the second embodiment of the present disclosure;

FIG. 13A is a graph showing changes in the amount of blood samples versus the yield of the manual centrifugal device in the second embodiment of the present disclosure;

FIG. 13B is a graph showing changes in the amount of blood samples versus the purity of the manual centrifugal device in the second embodiment of the present disclosure;

FIG. 13C is a graph showing changes in the blood sample volume of the manual centrifuge device in the second embodiment of the present disclosure versus the plasma volume obtained by the separation;

FIG. 14 is a comparison scatter diagram of the concentration of the AIDS p24 recombinant protein (HIV-1 p24) antigen and the color change between the manual centrifugal device in the second embodiment of the present disclosure and the standard solution;

FIG. 15 is a graph showing changes in concentration of the AIDS p24 recombinant protein (HIV-1 p24) antigen and the recovery rate by the manual centrifugal device in the second embodiment of the present disclosure;

FIG. 16 is a scatter diagram of the centrifugation time versus the yield for each of the angle arrangement between the blood storage tubes and the radial direction of the rotating shaft of the manual centrifugal device in the third embodiment of the present disclosure;

FIG. 17 is a scatter diagram of the centrifugation time versus the yield for each of the rotation speeds of the manual centrifugal device in the third embodiment of the present disclosure;

FIG. 18 is a scatter diagram showing changes in the position of the blood storage tube arrangement versus the yield of the manual centrifugal device in the third embodiment of the present disclosure;

FIG. 19 is a scatter diagram of the centrifugation time versus the yield performed with the manual centrifugal device in the third embodiment of the present disclosure according to different blood storage tube diameters;

FIG. 20 is a scatter diagram of the centrifugation time versus the yield for each rotation direction performed with the manual centrifugal device in the third embodiment of the present disclosure;

FIG. 21A is a schematic side view showing another aspect of a blood storage tube of the manual centrifugal device in the third embodiment of the present disclosure;

FIG. 21B is a scatter diagram of the centrifugation time versus the yield performed with the manual centrifugal device in the third embodiment of the present disclosure according to different vertical oblique angles of the blood storage tube with respect to the plane of the rotating platform;

FIG. 22 is a scatter diagram of the centrifugation time versus the yield performed with the manual centrifugal device in the third embodiment of the present disclosure according to each samplet with different HCT values;

FIG. 23A is a scatter diagram of the centrifugation time versus the rotation speed performed with the manual centrifugal device in the third embodiment of the present disclosure according to each operator selected by random; and

FIG. 23B is a scatter diagram of the centrifugation time versus the yield performed with the manual centrifugal device in the third embodiment of the present disclosure according to each operator selected by random.

### DETAILED DESCRIPTION OF THE EMBODIMENTS

The embodiments of the present disclosure are described by way of specific examples, and those skilled in the art can conceive understand the advantages and functions of the present disclosure from the present disclosure. The present disclosure may be implemented or applied by other different embodiments, and the each of details in the present specification may be variously modified and changed without departing from the spirit and scope of the present disclosure. In addition, all of the ranges and values herein are inclusive and combinable. Any value or point fallen within the ranges recited herein, such as any numerical value or point, may be the minimum or maximum value to derive the lower range and the like.

The present disclosure provides a manual centrifugal device for separating blood, including: a rotating platform; a rotating shaft disposed at the center of the rotating platform; and a plurality of blood storage tubes spaced apart from each other at a regular interval, disposed at a distance from the shaft, and restrictedly connected to the rotating platform. Accordingly, the rotating platform and the plurality of blood storage tubes rotate around the rotating shaft during a centrifugation operation. The term “restrictedly connected” used in the present disclosure means that a plurality of blood storage tubes are fixed to the rotating platform, or the plurality of blood storage tubes connected to the rotating platform can only be rotated or displaced within a restricted distance, for example, with a limiting member, such as a wire. When the rotating platform and the blood storage tube are connected, the displacement length of the blood storage tube does not exceed the length of the limiting member.

When a user operates the manual centrifugal device of the present disclosure, an external force is directly applied to the rotating platform or the rotating shaft until the rotating platform is rotated to a high rotation speed condition, and the centrifugal force field generated by the high-speed rotation is used for allowing the components of the sample placed in a blood storage tube to generate layered sedimentation by density.

The above operation does not require a power supply, and the intensity of centrifugal force generated is sufficient to cause the components of the blood sample in the blood storage tube to form layered sediments.

In another aspect, the centrifugal device of the present disclosure rotates horizontally relative to the plane of placement or causes the blood storage tube to appear less than 90 degrees horizontally, preferably less than 60, 45, 30 or 15 degrees. The rotation mode can eliminate the interference of gravity, as compared with the vertical rotation mode, and effectively improve the plasma yield and purity.

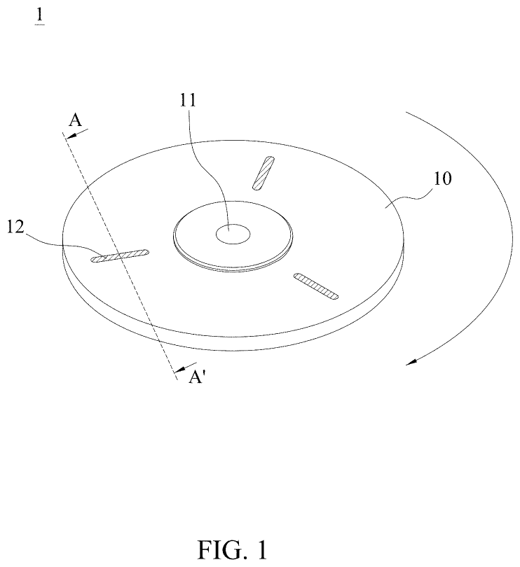

Referring to FIG. 1, FIG. 1 is a schematic view of a manual centrifugal device 1 of the present disclosure in a first embodiment. The manual centrifugal device 1 includes a rotating platform 10 and a plurality of blood storage tubes 12, wherein the plurality of blood storage tubes 12 accommodate the blood samples to be centrifuged, and are disposed on an upper surface of the rotating platform 10 at an equiangular interval around the rotating shaft 11.

The blood storage tube is a rod-like tube, and has a diameter of 1 to 10 mm; and the volume of each of the plurality of blood storage tubes is 1 to 1000 μL. By the viscous resistance of the blood sample in the thin tube diameter, the layers separated and settled are stabilized, and the flow between the components of the sample is suppressed.

In other embodiments, each of the plurality of blood storage tubes can have a diameter of 2, 3, 4, 5, 6, 7, 8, or 9 mm; and each of the plurality of blood storage tubes may have a volume of 1.5, 2, 2.5, 3, 3.5, 4, 4.5, 5, 5.5, 6, 6.5, 7, 7.5, 8, 8.5, 9, 9.5, 10, 12.5, 15, 17.5, 20, 30, 40, 50, 60, 70, 80, 90, 100, 200, 300, 400, 500, 600, 700, 800 or 900 μL.

The position where the blood storage tube is disposed on the rotating platform is preferably 1 to 20 cm from the rotating shaft.

In other embodiments, the distance between the plurality of blood storage tubes and the shaft can be 1.5, 2, 2.5, 3, 3.5, 4, 4.5, 5, 6, 7, 8, 9, 10, 11, 12, 13, 14, 15, 16, 17, 18 or 19 cm.

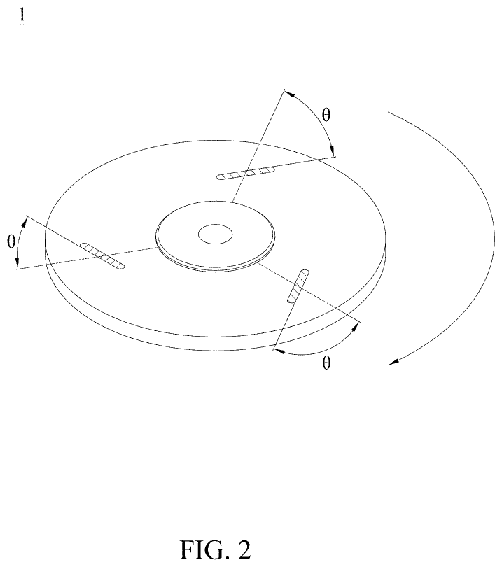

In one embodiment, as shown in FIG. 1, the plurality of the blood storage tubes are disposed along the radial direction of the rotating shaft. In another embodiment, as shown in FIG. 2, the blood storage tube is a straight tube disposed at an angle θ with respect to a radial direction of the rotating shaft, and the angle θ is less than 90 degrees, wherein the angle θ between the blood storage tube with respect to the radial direction of the rotating shaft is preferably in the range of 30 to 75 degrees.

In other embodiments, the angle between the blood storage tube and the radial direction of the rotating shaft can be 35, 40, 45, 50, 55, 60, 65 or 70 degrees, but is not limited thereto.

In another embodiment, the rotating platform is a plurality of rotating arms that are disposed at a regular interval along the radial direction of the rotating shaft and regularly distributed on the same plane.

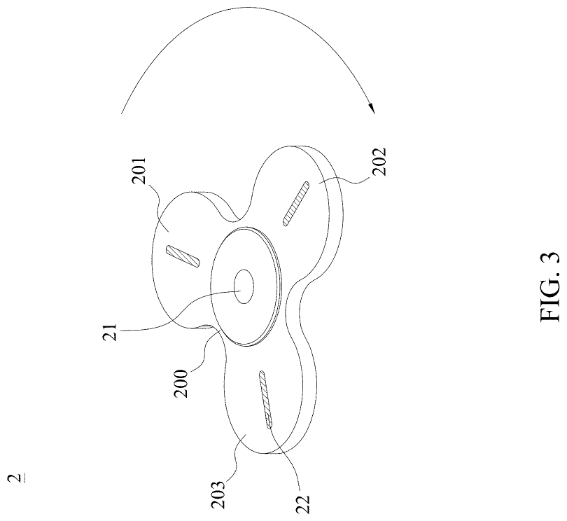

Referring to FIG. 3, FIG. 3 is a schematic view of a manual centrifugal device 2 of the present disclosure in a second embodiment. The rotating platform includes a connecting portion 200 for a rotating shaft 21 to be disposed thereon, and a plurality of arms 201, 202, and 203 disposed at a regular interval along the radial direction of the rotating shaft 21 and connected to the connecting portion 200; and a plurality of blood storage tubes 22 are disposed on the upper surfaces of the plurality of arms 201, 202, and 203.

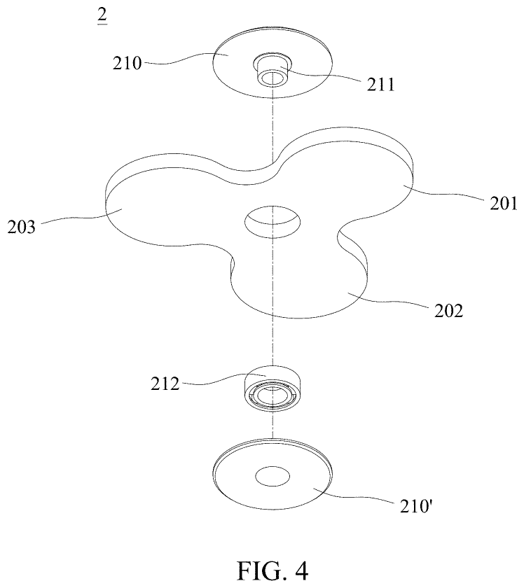

In a specific embodiment, as shown in FIG. 4, the rotating shaft 21 is provided with a rolling bearing 212 and two supporting members 210, 210′, and the supporting members 210 is provided with a stud 211. Thus, the stud 211 is passed through and fixed to the center of the rolling bearing 212, and is connected to the supporting member 210′. Through the rolling bearing 212, when the user applies an external force to the rotating arms 201, 202, and 203, the rotating arms 201, 202, and 203 can be continuously rotated by inertia, so as to generate a centrifugal action for the blood in the blood storage tube.

The rolling bearing includes an inner ring, an outer ring, and a plurality of rolling elements between the outer ring and the inner ring, and a cage that separates the plurality of rolling elements, wherein the rolling elements, the cage, the inner ring and the outer ring are made of low friction resistance materials.

In one embodiment, the load of the rotating arm is 50 to 500 grams to increase the rotational inertia and maintain the rotation time of the rotating arm.

In other embodiments, the load of the rotating arm may be 60, 70, 80, 90, 100, 200, 300 or 400 grams, but is not limited thereto.

In a specific embodiment, the blood storage tube is preferably disposed at a distance of 4 to 5 cm from the rotating shaft.

In one embodiment, the volume of the blood sample contained in the blood storage tube is 10 μL.

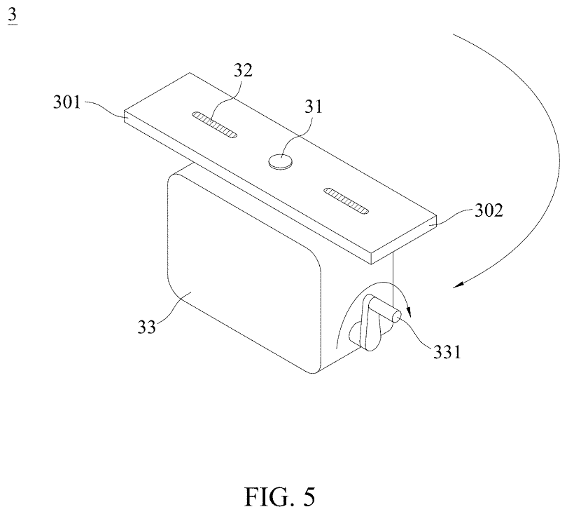

Please refer to FIG. 5, FIG. 5 is a schematic view of a third embodiment of a manual centrifugal device 3 of the present disclosure. The manual centrifugal device 3 includes a plurality of rotating arms 301, 302 disposed on the same plane at a equiangular interval in the radial direction; a rotating shaft 31 at the center of the plurality of rotating arms; a plurality of storage tubes 32 disposed on the upper surfaces of the plurality of rotating arms; and a base 33 including a crank handle 331 disposed on the outer casing of the base 33.

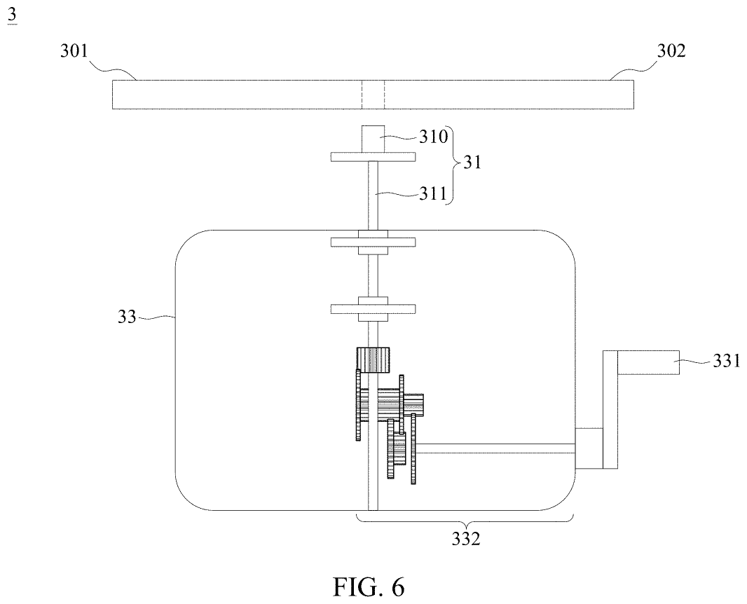

In a specific embodiment, as shown in the internal structure diagram of FIG. 6, the rotating shaft 31 is a shaft center 311 having a sliding bearing 310, which supports and drives the plurality of rotating arms 301 and 302. A gear transmission mechanism 332 is mounted in the casing cavity of the base 33, and the gear transmission mechanism 332 is connected to the rotation shaft 31 and the crank handle 331 on the outer casing.

When the user operates the manual centrifugal device of the present disclosure by rotating the crank handle 331, the rotating shaft 31 and the plurality of rotating arms 301,302 connected to the rotating shaft 31 are driven by the gear transmission mechanism 332 to rotate the rotating platform. The centrifugal force field generated at a high rotation speed causes layered sedimentation of the sample placed in the blood storage tube by the density.

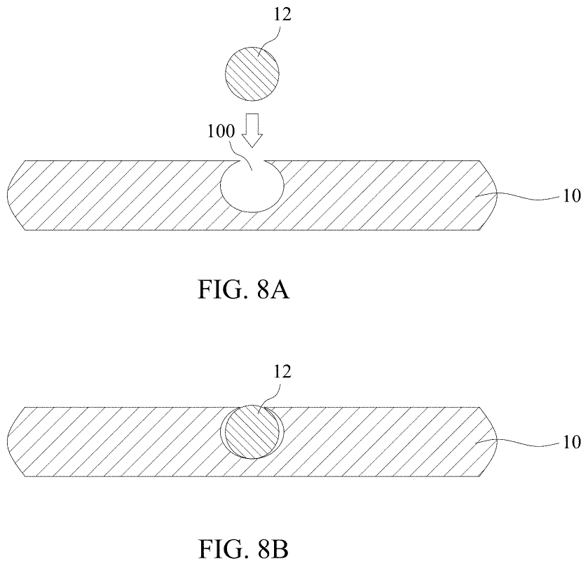

The fixing manner of the plurality of blood storage tubes on the upper surface of the rotating platform or the rotating arm, as shown in FIGS. 8A to 8B, is by forming a buckle groove 100 having convex ends on both sides and corresponding to the blood storage tube 12 on the upper surface of the rotating platform 10, to embed the plurality of blood storage tubes 12 into the surface of the rotating platform, so as to avoid displacement during centrifugation. Certainly, the fixing manner and material are not limited thereto.

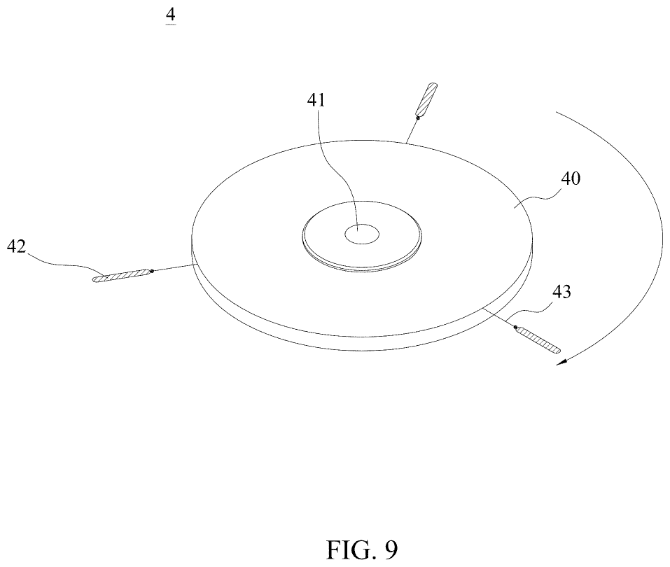

Referring to FIG. 9, FIG. 9 is a schematic view of a fourth embodiment of a manual centrifuge device 4 of the present disclosure. The manual centrifuge device 4 includes a rotating platform 40; a rotating shaft 41 disposed at the center of the rotating platform 40; and a plurality of blood storage tubes 42, wherein the plurality of blood storage tubes 42 receive the blood sample to be centrifuged and are fixed to the outer edge of the rotating platform 40 via a wire 43 at an equiangular interval around the rotating shaft 41.

The wire 43 is required to have a certain strength to avoid breakage during the rotation or impact of the blood storage tube, and the blood storage tube 42 may be fixed by an adhesive or directly tied with the wire 43.

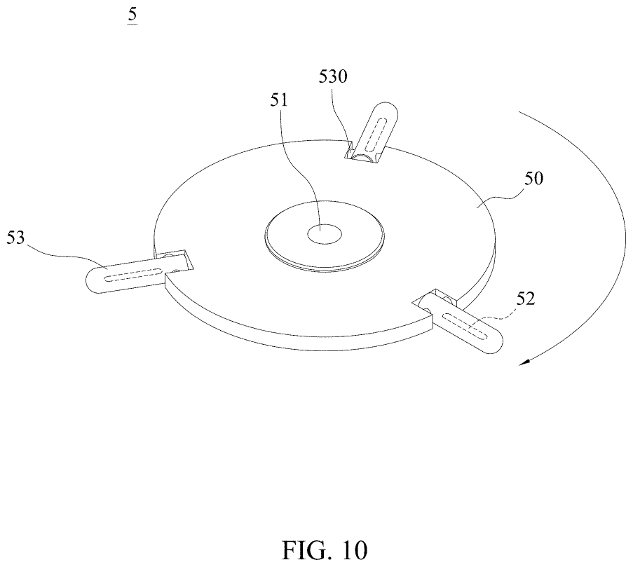

Referring to FIG. 10, FIG. 10 is a schematic view of a fifth embodiment of a manual centrifugal device 5 of the present disclosure. The manual centrifugal device 5 includes a rotating platform 50; a rotating shaft 51 disposed at the center of the rotating platform 50; a plurality of blood storage tubes 52 having the capacity for a blood sample to be centrifuged;

and a plurality of sleeves 53 provided to an outer edge of the rotating platform 50 to accommodate the plurality of blood storage tubes 52.

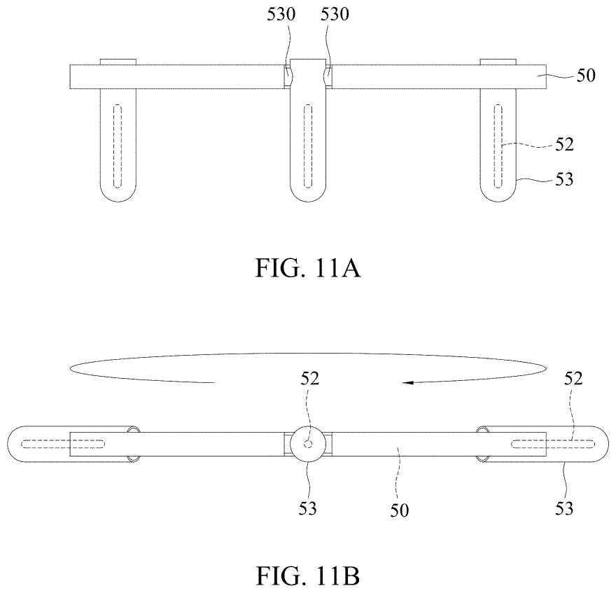

In a specific embodiment, as shown in FIG. 10, the sleeve 53 is pivotally connected with the outer edge of the rotating platform 50 at a position between the end thereof and the center of the sleeve 53. That is, the sleeve 53 is pivotally connected with the end of the sleeve 53 to a length less than one-half. As shown in FIGS. 10, 11A to 11B, the sleeve 53 is provided with a pivoting portion 530, and is disposed at two sidewalls between the end of the sleeve. Thus, the sleeve 53 is pivotally connected with the rotating platform 50, and the sleeve 53 is rotated relative to the pivoting portion 530. Certainly, in different implementations, the pivoting portion 530 can also be disposed on the rotating platform 50.

When the manual centrifuge device 5 of the present disclosure is stationary, the sleeve 53 and the blood storage tube 52 therein can be pivoted by gravity to be perpendicular or nearly perpendicular to the rotating platform 50. During centrifugation, the centrifugal force field generated by the high speed rotation causes the sleeve 53 to pivot to be in a plane with the rotating platform 50, or to rotate the blood storage tube and the plane of the rotating platform 50 by less than 90 degrees, and preferably less than 60, 45, 30 or 15 degrees.

In addition, the rotating platform and the rotating arm can be made of materials such as plastic, wood and metal, and are not limited thereto. The rotating platform is preferably made of a plastic material, and is further adjusted according to actual needs, so that the manual centrifugal device of the present disclosure is easy to carry without being restricted by the operating location.

The present disclosure further provides a method of using the manual centrifugal device described above, including the steps of: loading blood into at least one of the blood storage tubes; in case of the blood storage tube with an opening, optionally closing the opening of the blood storage tubes; restrictedly connecting the blood storage tube to the rotating platform; and spinning the rotating platform of the centrifuge device manually to separate blood plasma and blood cells in the blood storage tube.

In a specific embodiment, when the rotating shaft includes a rolling bearing and a supporting member, the manual centrifugal device rotates at a speed of 1000 to 1200 rpm, and the separation operation takes 3 to 15 minutes; wherein, the blood storage tubes fix to the upper surface of the rotating platform, and the supporting member includes a stud fixed through the center of the rolling bearing.

In other embodiments, the manual centrifugal device can have a rotation speed of 1050, 1100, or 1150 rpm, but is not limited thereto; the separation operation can take 4, 5, 6, 7, 8, 9, 10, 11, 12, 13 or 14 minutes, but is not limited to this.

In another embodiment, when the rotating shaft is a shaft center of a sliding bearing, the rotating speed of the centrifugal device is a speed of 2000 to 3000 rpm, so that the blood storage tube is fixed to the upper surface of the rotating platform, and the separation time required is 1.5 to 5 minutes.

In other embodiments, the manual centrifugal device can have a rotation speed of 2100, 2200, 2300, 2400, 2500, 2600, 2700, 2800, or 2900 rpm, but is not limited thereto. It can be 2, 2.5, 3, 3.5, 4 or 4.5 minutes, but is not limited thereto.

Since the centrifugal device of the present disclosure centrifugally separates blood cells and plasma in the blood by manually driven, the separation efficiency, the yield and the purity can all meet the requirements of the detection accuracy, and is more convenient than the existing centrifugal equipment. Simple operation and rapid detection can greatly reduce the medical cost burden in the low-resource areas.

The present disclosure further provides a system for analyzing a target protein in plasma of a patient in a low-resource area, and the system includes the above-described manual centrifugal device for obtaining plasma containing the target protein; and a trace detection device with a captured protein, wherein the captured protein specifically binds to the target protein in the plasma.

After centrifugation by the manual centrifugal device, the plasma and blood cells in the blood are sedimented and layered, and the user breaks the blood storage tube along the stratified position, and takes the plasma portion as a sample liquid. The sample fluid is in direct contact with the trace detection device, so that the captured protein specifically binds to the target protein in the plasma. The enzyme-labeled protein and the substrate are sequentially added to give the conjugate a predetermined color; and the color change test results are obtained.

The target protein refers to a protein detectable by a detection device, including an antibody or an antigen. The captured protein is a protein that can specifically bind to a conjugate of a target protein to specifically recognize the target protein. In a specific embodiment, the target protein reacts with the surface of the trace detection device having the captured protein to form an irreversible chemical bond in a short time, so that the target protein is not easily desorbed.

In a specific embodiment, the enzyme-labeled protein can be an antibody with horseradish peroxidase (HRP), and the substrate capable of undergoing an enzyme reaction with horseradish peroxidase can be one selected from the group consisting of potassium iodide (KI), 3,3′,5,5′-tetramethylbenzidine (TMB), 3,3′-diaminobenzidine (DAB), 2,2-diazo-bis(3-ethyl-benzothiazoline-6-sulfonic acid) diammonium salt (ABTS) and phosphorus phenylenediamine dihydrochloride (OPD).

In a specific embodiment, the target protein is an antigen, the captured protein is an antibody, the enzyme-labeled protein can be an antibody with horseradish peroxidase (HRP), and the substrate is 3,3′,5,5′-tetramethylbenzidine (TMB).

In one embodiment, the trace detection device has a minimum detection limit of 0.1 ng/ml and a detection time of 7.1 minutes.

As can be seen from the above, the centrifugal device of the present disclosure is combined with a trace detection device, which has the advantages of rapidity and low cost, and is popular for use in developing countries where medical resources are scarce.

The present disclosure will be further described in detail below by way of examples.

### Example 1-1 of a Manual Centrifugal Device in a Second Embodiment: Test on Changes in Blood Storage Tube Position

First, about 3 μL of a blood sample was taken and dropped onto a glass slide, and the number of blood cells in the blood sample was counted with a whole blood image.

Next, a plastic tube having a length of 50 mm and an inner diameter of 1 mm was used as a blood storage tube, and 10 μL of the blood sample was taken through a suction device at one end of the blood storage tube, and the blood storage tube was filled. Both of the end openings of the blood storage tube were heated after filling until melting, and the pliers were pinched to seal the openings of the blood storage tube.

After the blood sample was loaded into the plurality of blood storage tubes, the sealed blood storage tube was placed in the buckle groove of the manual centrifugal device in the second embodiment, and the blood storage tube was embedded and fixed to the upper surface of the centrifugal device. The blood storage tubes were disposed along the radial direction of the rotating shaft, as shown in FIG. 3, wherein the rotation radius of the manual centrifugation refers to the distance between the center of the blood storage tube and the rotating shaft.

In this example, the blood storage tubes were provided with rotation radius of 30 mm, 35 mm, 40 mm, 45 mm, and 50 mm, respectively.

An external force was applied to the rotating arms of the manual centrifugal device by hand, so that the rotating arms of the centrifugal device and the blood storage tubes thereon reach a maximum rotational speed of 1200 rpm; and after 10 to 15 minutes of rotation, blood separation was completed.

The blood storage tube was taken out, the blood storage tube was broken along the stratified position, the plasma was partially dropped onto a glass slide, the number of blood cells in the plasma was counted by using an optical microscope, and the plasma purity was obtained according to the following equation:

$$Purity ( \% ) = ( 1 - \frac{Blood cell number in plasma image}{Blood cell number in whole blood image} ) \times 100 \%$$

Furthermore, the image length of the plasma and blood in the blood storage tube was intercepted by ImageJ, and the yield was obtained according to the following equation:

$$\begin{matrix}Yield ( \% ) = \frac{Plasma volume}{Whole blood volume} \times 100 \% \\ = \frac{Plasma image length}{Whole blood image length} \times 100 \%\end{matrix}$$

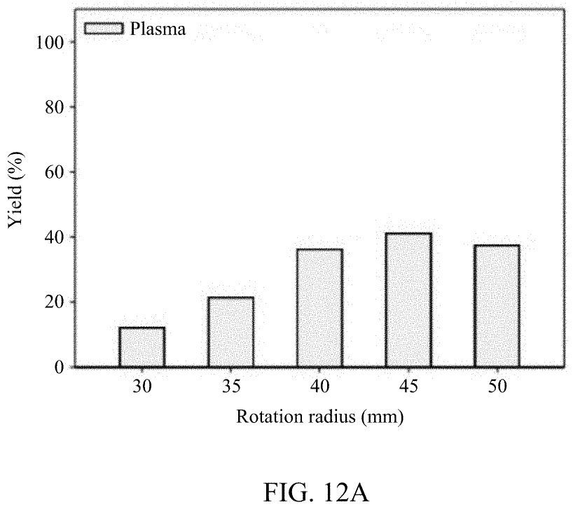

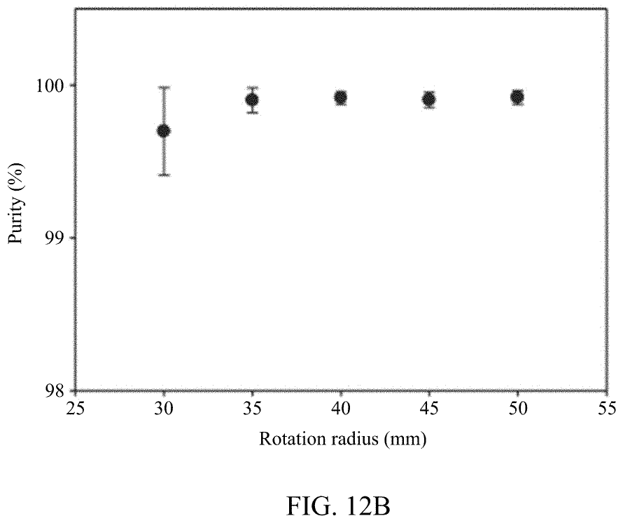

Referring to FIGS. 12A and 12B, FIGS. 12A and 12B are diagrams showing changes in the position of blood storage tube arrangement versus yield and purity according to the present embodiments. When the rotation radius got larger, the plasma yield became higher; and when the rotation radius exceeds 4 cm, the yields thereof were all higher than 30%, though the yield change has a tendency to gradually slow down. However, in the range of the rotation radius of 30 to 50 mm, the purity of the plasma obtained by the separation was more than 99%. Obviously, the manual centrifugal device of the present disclosure has an excellent function of plasma separation.

### Example 1-2 in the Second Embodiment of the Manual Centrifugal Device: Test on Changes in Blood Sample Amount

The treatment method in Example 1-2 was the same as in Example 1-1, except that the blood storage tube was disposed at a rotation radius of 40 mm, and the volumes of the blood samples were 5 μL and 7.5 μL, 10 μL, 12.5 μL, 15 μL, respectively. The effect of changes in blood sample volume on the yield, purity, and plasma volume obtained from separation was evaluated.

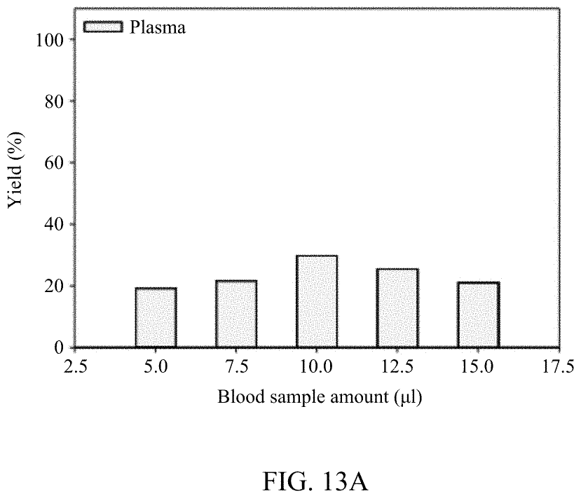

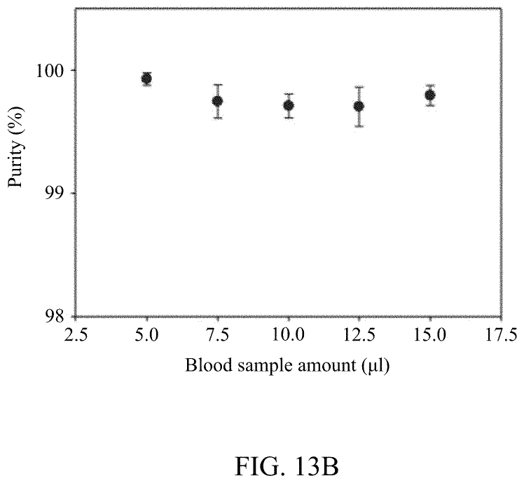

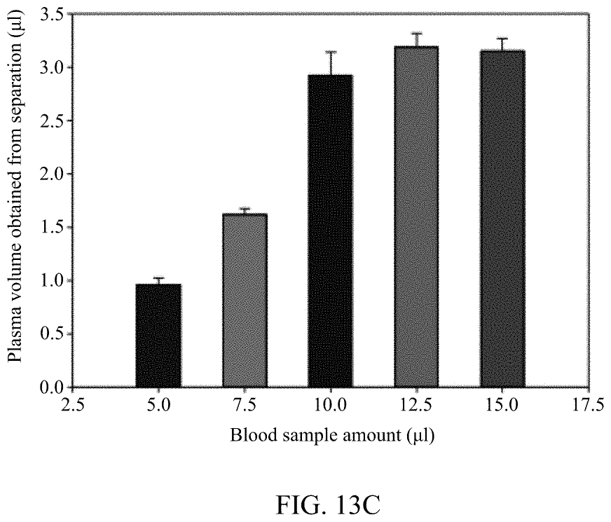

Referring to FIGS. 13A to 13C, FIGS. 13A to 13C are diagrams showing changes in the yield, purity, and plasma volume obtained from separation according to the example, and the results of the experiment show that the blood sample has a higher separation yield value at 10 μL. When the amount of blood sample exceeded 10 μL, the plasma volume obtained from separation was about 3 μL, which was sufficient for trace detection. In the range of 5 to 15 μL of blood sample, the plasma purity obtained by the separation was more than 99%. Obviously, the manual centrifugal device of the present disclosure has an excellent function of plasma separation.

### Example 1-3 in the Second Embodiment Manual Centrifugal Device: Test for Separation Plasma for Trace Detection Test

Preparation of a Paper-Based Detection Platform:

a cellulose test paper was used as a substrate, and a solid hard wax was printed onto the test paper by a wax printer to form hole pattern, and the wax block was heated by using a heat plate at 110° C. for 15 minutes to melt. After re-solidification, a circular area having a hole diameter of about 5 mm was formed.

Next, the substrate was thermostated at 40° C., and 3 μL of mouse AIDS p24 recombinant protein antibody (Mouse anti-HIV-1 p24) was injected as a captured protein at the hole position, and dried at 40° C. for 1 minute. The mouse AIDS p24 recombinant protein antibody was immobilized on the surface of the substrate, wherein the mouse AIDS p24 recombinant protein antibody solution was diluted with a phosphate buffer solution (PBS) to a concentration of 2 μg/ml.

The 5% bovine serum albumin (BSA) solution diluted with 3 μL of PBS covered a portion of the unbounded active site, and baked at 40° C. for 1 minute to obtain a mouse AIDS p24 recombinant protein antibody-immobilized substrate surface.

Detection for Standard Solution Test:

a blank solution and seven standard solution of 3 μL with a concentration of AIDS p24 recombinant protein (HIV-1 p24) antigen of 0.03 to 30 ng/ml prepared at 25° C., respectively. The above solutions gradually injected onto paper-based detection platform prepared above and baked at a temperature of 40° C. for 1 minute.

Next, 3 μL of 0.64 μg/ml of PBS-diluted Mouse anti-HIV-1 p24 labeled with HRP was introduced and dried at 40° C. for 2 minutes to be immersed in a phosphate buffer solution containing Tween 20 as a washing buffer solution for 30 seconds, so as to the enzyme-labeled protein (Mouse anti-HIV-1 p24-HRP) specifically bind to the target protein (HIV-1 p24) to form a conjugate.

After washing and drying at 40° C. for 7 minutes, 3 μL of TMB was introduced for an enzymatic reaction for 10 minutes; and then a microscope was used for capturing images, and color changes was analyzed with an image analysis software.

Detection of the Plasma in the Second Embodiment of the Manual Centrifugal Device:

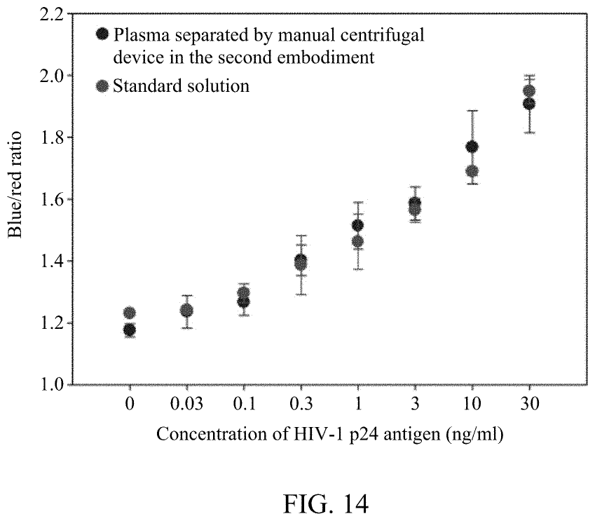

the treatment method was the same as in the above standard solution detection test, except that the standard solution was changed to the plasma obtained by the centrifugal separation method of Example 1-2, wherein the amount of the blood sample was 10 μL, and the blood samples each contained a blank solution and seven solutions with HIV-1 p24 antigens at a concentration of 0.03 to 30 ng/ml; and the test curve of Example 1-2 was compared with that of the standard solution, and is shown in FIG. 14.

Referring to FIG. 14, FIG. 14 is a comparison scatter diagram of the concentration of the AIDS p24 recombinant protein antigen and color change in the example. It can be seen from the experimental results that the dynamic linear range of plasma (optimal detection range) containing the recombinant protein antigen containing AIDS p24 separated by the second embodiment of the centrifugal device of the example was 0.1 to 10 ng/ml (R²=0.9852), and the minimum detection limit (LOD) was 0.03 ng/ml. The results almost overlap with those of the standard solution. Obviously, the centrifuged plasma of the manual centrifugal device of the present disclosure has an excellent detection accuracy and is indeed applicable.

Recovery Rate of Plasma in the Manual Centrifugal Device of Second Embodiment:

The “recovery rate” was used to compare the differences between the standard plasma and plasma separated from the manual centrifugation device in the second embodiment to understand the interference to the target protein by the plasma matrix obtained by the manual centrifugation device in the second embodiment.

The recovery rate was calculated according to the following equation:

$$R \% = \frac{( SSR - SR )}{SA} \times 100 \%$$

wherein the R value denotes the recovery rate; the SSR value denotes the concentration value obtained by separating the plasma from the manual centrifugal device in the second embodiment, adding the AIDS p24 recombinant protein (HIV-1 p24) antigen, and then detecting by the above-mentioned paper-based detection platform; the SR value denotes the measured concentration value without adding the HIV-1 p24 antigen (in the present example, it is 0); and the SA value denotes the concentration value after adding the HIV-1 p24 antigen.

The method for measuring the recovery rate was the same as the treatment method in the above Examples 1-2, except that after plasma separation, additional concentrations of the AIDS p24 recombinant protein (HIV-1 p24) antigen of 0.1 ng/ml, 1 ng/ml, and 10 ng/ml were added to the plasma, and the concentration value was then obtained by the above-mentioned paper-based detection platform. The detection method was the same as the test method for the above-mentioned manual centrifugal device in the second embodiment.

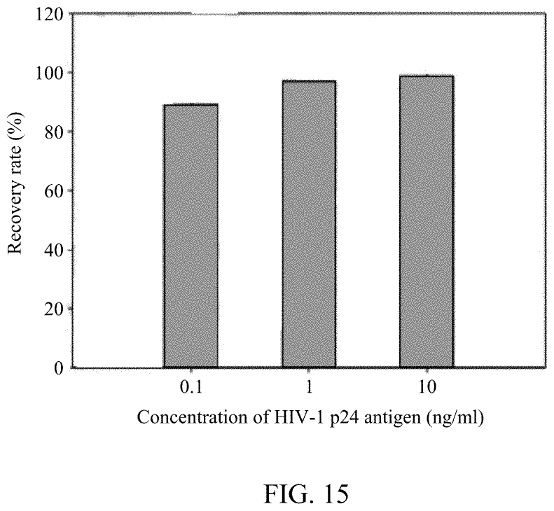

Referring to FIG. 15, FIG. 15 is a graph showing changes in antigen concentration of AIDS p24 recombinant protein and recovery rate by the manual centrifugal device in the second embodiment. From the experimental results, the recovery rate of the centrifugal device in the second embodiment of the present disclosure was obtained between 80 and 100%; when the concentration was high, the recovery rate was even more than 97%. Therefore, the plasma obtained by the centrifugal device in the second embodiment of the present disclosure has an excellent plasma quality; also, such device exhibits excellent performance in sensitivity test for medical trace detection.

### Example 2-1 Manual Centrifugal Device in the Third Embodiment: Test on Changes in Oblique Angle of the Blood Storage Tube with Respect to the Radial Direction

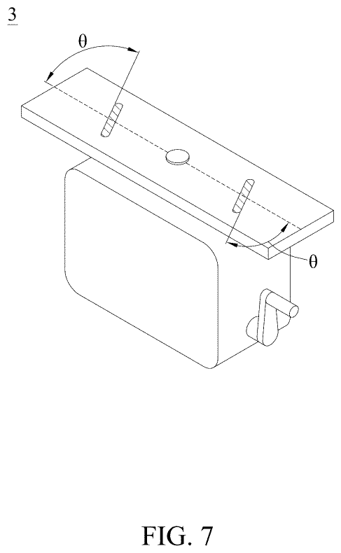

The treatment method in Example 2-1 was the same as in Example 1-1, except that the manual centrifugal device in the second embodiment was replaced with the manual centrifugal device in the third embodiment, and the blood storage tube was set to have a rotation radius of 15 mm. The blood storage tube was disposed at an angle θ with respect to the radial direction. As shown in FIG. 7, the rotation speed of the rotating arm of the centrifugal device and the blood storage tube thereon was adjusted to 2200 rpm, and the plasma was separated by observing different angles θ. The yield varied with the centrifugation times.

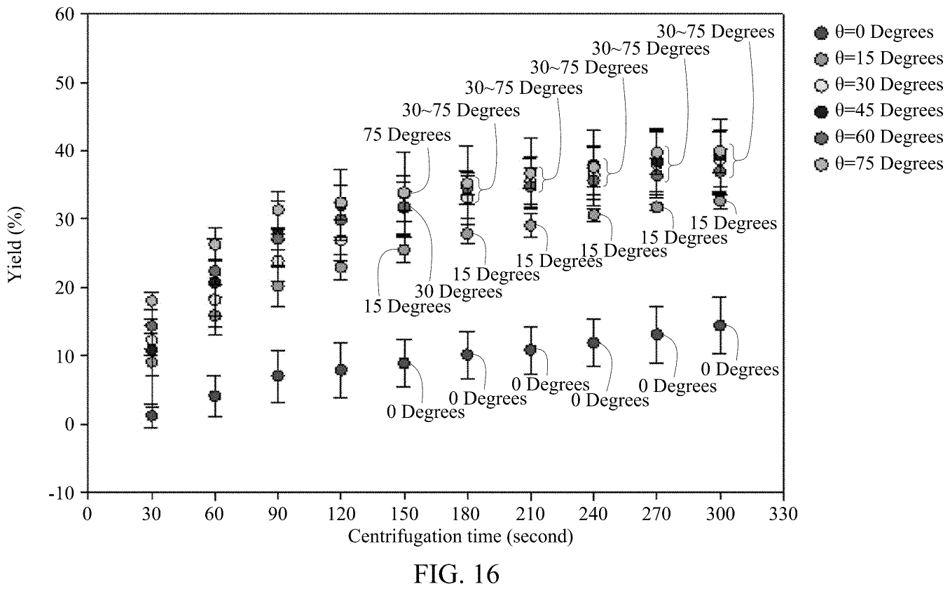

Referring to FIG. 16, FIG. 16 is a scatter diagram of the centrifugation time versus yield for each of angle arrangement between the blood storage tubes and the radial direction of the rotating shaft of the manual centrifugal device according to the embodiments. It can be seen from the experimental results that when the angle between the blood storage tubes and the radial direction was 30 to 75 degrees, the yield can be more than 30% at 150 seconds. Obviously, the angle θ of the blood storage tube of the manual centrifugal device of the present disclosure indeed have an excellent effect of improving the plasma separation efficiency with respect to the arrangement of along the radial direction.

### Example 2-2 of a Manual Centrifugal Device in a Third Embodiment: Test on Changes in Rotation Speeds

The treatment method in the example 2-2 was the same as in the example 2-1, except that the blood storage tube was set to have a rotation radius of 25 mm, and the blood storage tube was disposed at an angle θ of 60 degrees with respect to the radial direction. With respect to different rotation speeds, variations of the yield of the separated plasma with the centrifugation time were observed.

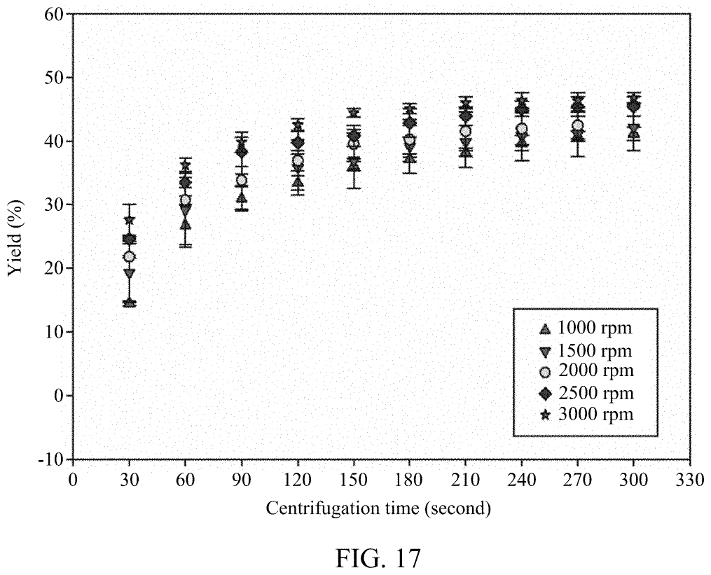

Referring to FIG. 17, FIG. 17 is a scatter diagram of centrifugation time versus yield for each of rotation speed according to the embodiment, and the experimental results showed that the higher the rotation speed, the shorter the amount of time high plasma separation yield can be achieved. The plasma separation efficiency was high, and the yield was also high, though the yield to gradually decreased with time. Therefore, the blood storage tube was disposed at an angle θ, and with the adjustment of high centrifugal speed, the plasma can reach a yield of more than 40% in 90 seconds, which has the effect of improving plasma separation efficiency.

### Example 2-3 of a Manual Centrifugal Device in a Third Embodiment: Test on Changes in Positions of Blood Storage Tubes

The treatment method in the example 2-3 was the same as in the example 2-1, except the blood storage tubes were disposed at an angle θ of 0 degree and 30 degree with respect to the radial direction, and blood separation was completed after 150 seconds of rotation. In this example, the blood storage tubes were disposed with rotation radius of 15 mm, 25 mm, 35 mm, and 45 mm, respectively. With respect to different rotation radius, variations of the yield of the separated plasma with different angle θ were observed.

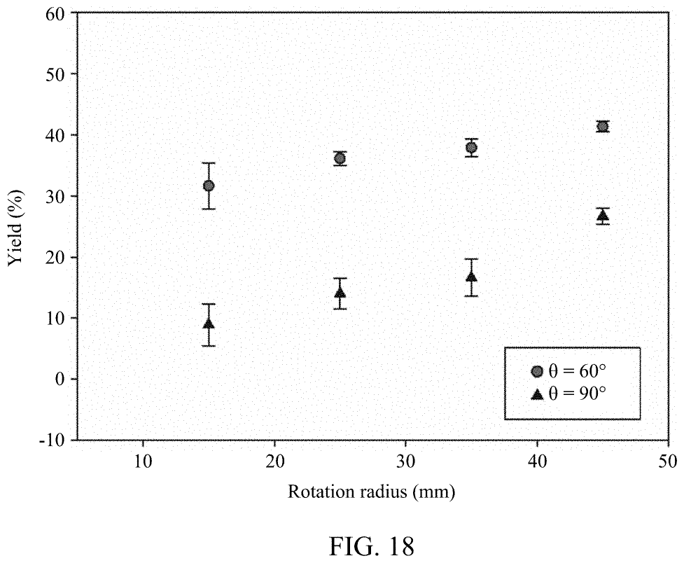

Referring to FIG. 18, FIG. 18 is scatter diagrams showing changes in the position of blood storage tube arrangement versus yield according to the present embodiments. When the rotation radius got larger, the plasma yield became higher, and the trend is identical at the angle θ of 30 degree in the range of the rotation radius of 15 to 45 mm. In addition, the bigger the angle θ, the higher the plasma yield. Obviously, the angle θ of the blood storage tube of the manual centrifugal device of the present disclosure indeed have the same effect of improving the plasma separation efficiency with respect to the arrangement of rotation radius.

### Example 2-4 of a Manual Centrifugal Device in a Third Embodiment: Test on Changes in Blood Storage Tube Diameter

The treatment method in the example 2-4 was the same as in the example 2-1, except the blood storage tube was disposed at an angle θ of 30 degree with respect to the radial direction, and the blood storage tubes were set to have a rotation radius of 25 mm. In this example, the blood storage tube diameters (d) were provided with 1 mm, 1.3 mm, and 1.8 mm, respectively. With respect to centrifugation time, variations of the yield of the separated plasma with different blood storage tube diameter (d) were observed.

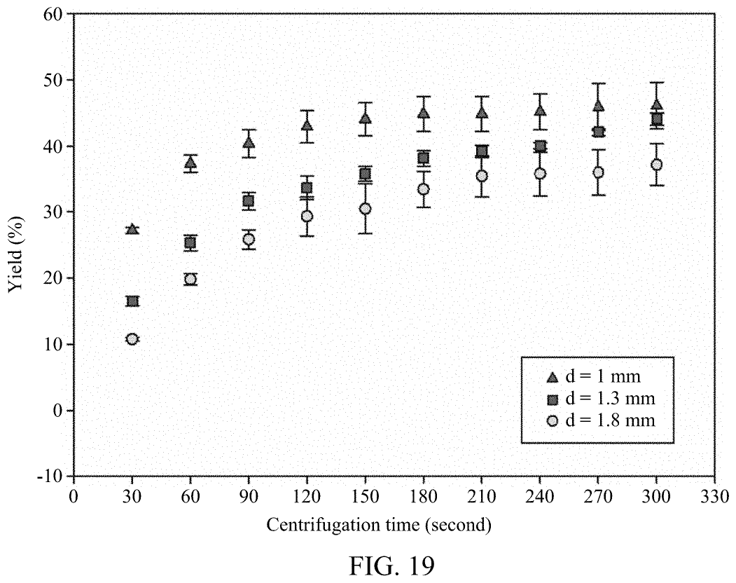

Referring to FIG. 19, FIG. 19 is a scatter diagram of the centrifugation time versus yield for each blood storage tube diameter according to the embodiments. It can be seen from the experimental results that the blood storage tube diameter of 1 mm was used, the plasma can reach a yield of more than 40% in 90 seconds, which has the better effect of improving plasma separation efficiency. In addition, the smaller the blood storage tube diameter, the higher the plasma yield. Therefore,

### Example 2-5 of a Manual Centrifugal Device in a Third Embodiment: Test on Effect of Rotation Direction

The treatment method in the example 2-5 was the same as in the example 2-1, except the blood storage tube was disposed at an angle θ of 30 degree with respect to the radial direction, and the blood storage tube was set to have a rotation radius of 25 mm Variations of the yield of the separated plasma with different rotation direction and centrifugation time were observed.

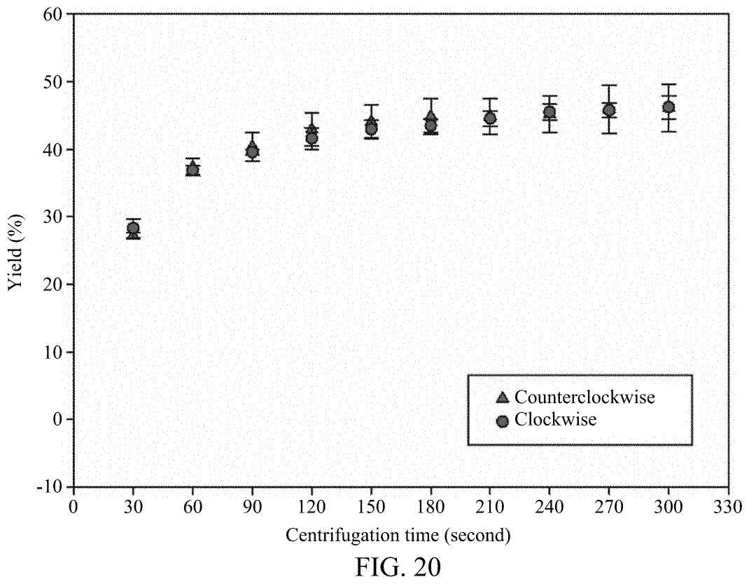

Referring to FIG. 20, FIG. 20 is a scatter diagram of the centrifugation time versus yield for each rotation direction according to the embodiments. It can be seen from the experimental results that the rotation direction has little effect on the yield of plasma separation. Therefore, the separation efficiency would not be affected by the rotation direction.

### Example 2-6 of a Manual Centrifugal Device in a Third Embodiment: Test on Changes in Vertical Oblique Angle of the Blood Storage Tube with Respect to the Plane of the Rotating Platform

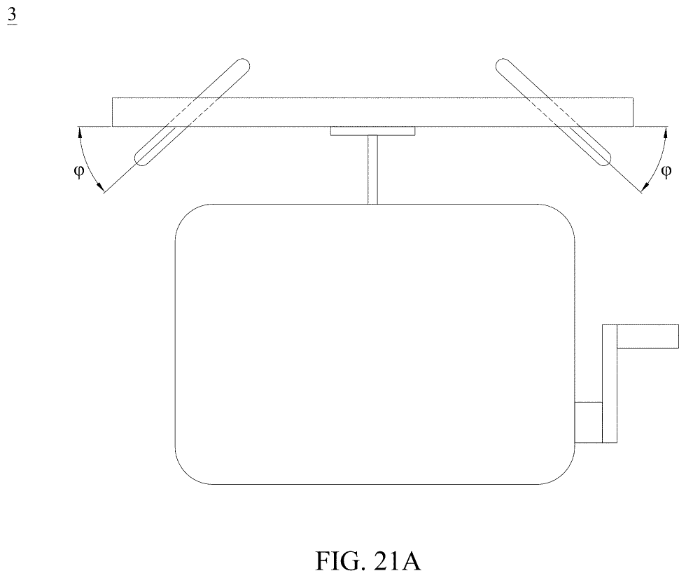

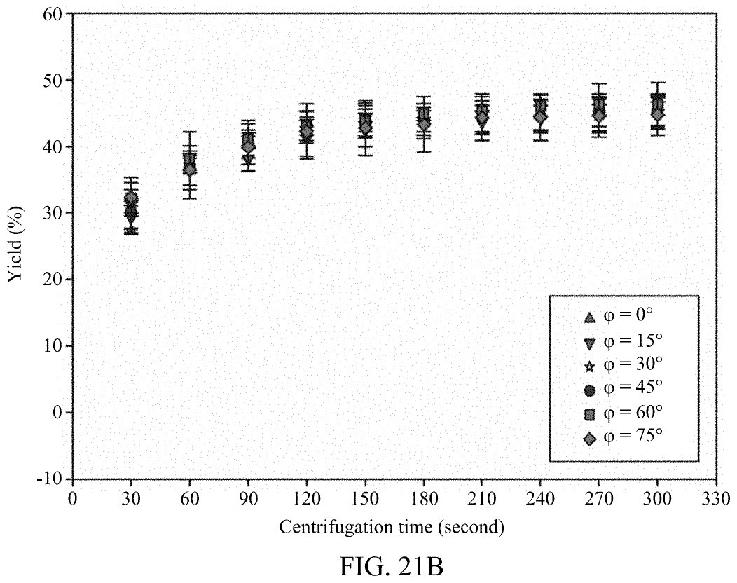

The treatment method in Example 2-6 was the same as in Example 2-5, except that the blood storage tube was fixed at an vertical oblique angle φ with respect to the plane of rotating platform of manual centrifugal device in the third embodiment (as shown in FIG. 21A). In this example, the vertical oblique angle φ of the blood storage tube were provided with 0°, 15°, 30°, 45°, 60°, and 75°, respectively. With respect to centrifugation time, variations of the yield of the separated plasma with different vertical oblique angle φ were observed

Referring to FIG. 21B, FIG. 21B is a scatter diagram of the centrifugation time versus yield for each test with different vertical oblique angle φ according to the embodiments. It can be seen from the experimental results that tilting the blood storage tube with vertical oblique angle φ with respect to the plane of rotating platform has little effect on the result of plasma separation. Therefore, the separation efficiency would not be affected by tilting the blood storage tubes to each vertical oblique angle.

### Example 2-7 of a Manual Centrifugal Device in a Third Embodiment: Test on Effect of Samples' Hematocrit (HCT)

The treatment method in the example 2-7 was the same as in the example 2-5. In this example, each of the samples has different hematocrit (HCT) status. The hematocrit (HCT) value means the ratio of red blood cells in the blood measured by an automated analyzer. The HCT value of samples in this example were selected as 45.3%, 47.9%, and 48.3%, respectively. With respect to centrifugation time, variations of the yield of the separated plasma from different sample's HCT were observed.

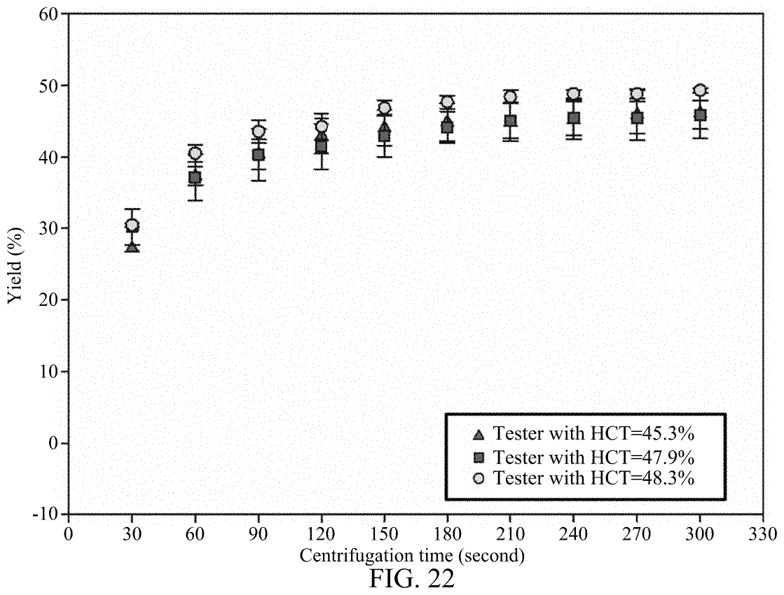

Referring to FIG. 22, FIG. 22 is a scatter diagram of the centrifugation time versus yield for each sample with different HCT value according to the embodiments. It can be seen from the experimental results that the HCT status of samples has little effect on the result of plasma separation. Therefore, the separation efficiency would not be affected by the source of sample.

### Example 2-8 of a Manual Centrifugal Device in a Third Embodiment: Test on Effect of Operation with Different Operators

The treatment method in the example 2-8 was the same as in the example 2-5. In this example, the operator was selected by random to determine whether the separation result would be affected by different operator. With respect to centrifugation time, variations of the rotation speed of centrifugal device and the yield of the separated plasma were observed.

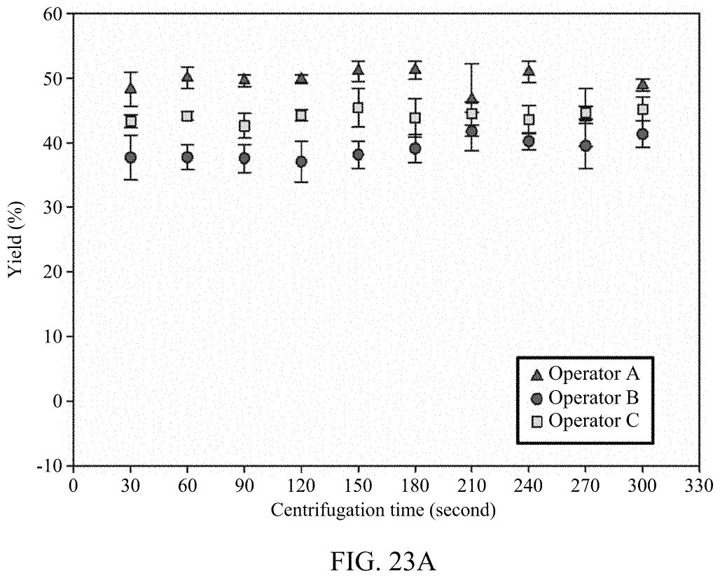

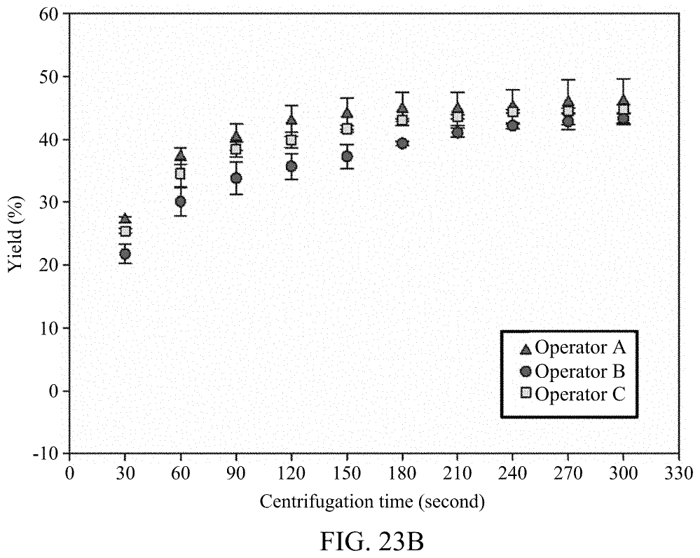

Referring to FIG. 23A and FIG. 23B, FIG. 23A and FIG. 23B are scatter diagram of the centrifugation time versus rotation speed and yield for each operator according to the embodiments, respectively. It can be seen from the experimental results that the status of operator has little effect on the result of plasma separation. Therefore, the separation efficiency maintains stable and is not affected by different operator.

In conclusion, the centrifugal device of the present disclosure was manually driven to centrifugally separate blood cells and plasma in the blood, thereby greatly reducing the medical cost burden of the low-resource area. Further, the centrifugation device of the present disclosure rotates horizontally relative to the plane of disposal, or rotates the blood storage tube with respect to the plane at less than angle of 90 degrees, preferably less than 60 degrees, 45 degrees, 30 degrees or 15 degrees. In comparison with a vertical rotation centrifuge, it can eliminate the interference of gravity, and improve the yield and purity of plasma, so as to meet the requirements of detection accuracy.

The above embodiments are merely illustrative, and are not intended to limit the present disclosure. Modifications and variations of the above-described embodiments can be made by those skilled in the art without departing from the spirit and scope of the present disclosure. Therefore, the scope of the present disclosure is defined by the scope of the appended claims. As long as the effects and implementation purposes of the present disclosure are not affected, they should be encompassed in the present disclosure.

What is claimed is:

1. A manual centrifugal device for separating blood, comprising:
a rotating platform comprising a plurality of rotating arms;
a rotating shaft disposed at a center of the rotating platform, with the plurality of rotating arms equidistantly disposed along a radial direction of the rotating shaft; and
a plurality of blood storage tubes disposed at a regular interval from one another, and restrictedly positioned on the rotating platform at a distance from the rotating shaft,
wherein the rotating platform and the plurality of blood storage tubes rotate around the rotating shaft during centrifugation.

2. The manual centrifugal device of claim 1, wherein the plurality of blood storage tubes are disposed on an upper surface of the rotating platform.

3. The manual centrifugal device of claim 2, wherein the rotating platform has a buckle groove, and the plurality of blood storage tubes are embedded in the buckle groove.

4. The manual centrifugal device of claim 2, wherein the plurality of blood storage tubes are disposed away from the rotating shaft at a distance of 1 cm to 20 cm.

5. The manual centrifugal device of claim 2, wherein the plurality of blood storage tubes are disposed along a radial direction of the rotating shaft.

6. The manual centrifugal device of claim 2, wherein the plurality of blood storage tubes are disposed at an angle of less than 90 degrees with respect to a radial direction of the rotating shaft.

7. The manual centrifugal device of claim 6, wherein the angle is from 30 degrees to 75 degrees.

8. The manual centrifugal device of claim 1, wherein the rotating platform is provided with a plurality of through holes for accommodating the plurality of blood storage tubes, and the plurality of blood storage tubes are disposed at an oblique angle of less than 90 degrees with respect to the plane of the rotating platform.

9. The manual centrifugal device of claim 1, wherein the rotating platform comprises a connecting portion for the rotating shaft, and the plurality of rotating arms are connected to the connecting portion.

10. The manual centrifugal device of claim 9, wherein the rotating arm has a load of 50 grams to 500 grams.

11. The manual centrifugal device of claim 1, wherein each of the blood storage tubes has a volume of 1 μL to 1000 μL.

12. The manual centrifugal device of claim 1, wherein the plurality of blood storage tubes have a diameter of 1 mm to 10 mm.

13. The manual centrifugal device of claim 1, wherein the rotating shaft comprises a rolling bearing and a supporting member having a stud, and the stud is fixed through the rolling bearing.

14. The manual centrifugal device of claim 1, wherein the rotating shaft is a sliding bearing.

15. The manual centrifugal device of claim 14, further comprising a base comprising a gear transmission mechanism and a crank handle connected to the gear transmission mechanism, and the rotation shaft is disposed on the base, and connected to the gear transmission mechanism.

16. A method of separating blood by using the manual centrifugal device of claim 1, comprising:
loading the blood into at least one of the blood storage tubes;
restrictedly positioning the blood storage tube to the rotating arms of the rotating platform; and
spinning the rotating platform of the manual centrifugal device manually to separate blood plasma and blood cells in the blood storage tube.

17. The method of claim 16, wherein the plurality of the blood storage tubes are fixed to an upper surface of the rotating platform.

18. The method of claim 17, wherein the rotating shaft comprises a rolling bearing and a supporting member having a stud, and the stud is fixed at a center of the rolling bearing, and wherein the manual centrifugal device has a rotation speed of 1000 rpm to 1200 rpm and a separation time of 3 minutes to 15 minutes.

19. The method of claim 17, wherein the rotating shaft is a shaft center of a sliding bearing, and the manual centrifugal device has a rotation speed of 2000 rpm to 3000 rpm and a separation time of 1.5 minutes to 5 minutes.

20. A system for analyzing a target protein in plasma of a patient, comprising:
the manual centrifugal device of claim 1 for obtaining plasma containing the target protein; and
a trace detection device with a captured protein, wherein the captured protein specifically binds to the target protein in the plasma.
